(19) United States
(12) Patent Application Publication
(10) Pub. No.: US 2020/0360654 A1
(43) Pub. Date: Nov. 19, 2020

(54) SYSTEM AND METHOD FOR A THERAPEUTIC BOX

(71) Applicant: Karen Hanlon GROVE, Belleair Beach, FL (US)

(72) Inventor: Karen Hanlon Grove, Belleair Beach, FL (US)

(21) Appl. No.: 16/875,818

(22) Filed: May 15, 2020

Related U.S. Application Data
(60) Provisional application No. 62/849,782, filed on May 17, 2019.

(51) Int. Cl. A61M 21/02; B44D 2/00

(52) U.S. Cl. CPC A61M 21/02; A61M 2021/0027; B44D 2/002

(57) ABSTRACT

The invention provides self-care boxes and methods for using the self-care boxes for purposes such as self-expression, relaxation, enhanced self-awareness, and self-discovery.

### CROSS REFERENCE TO RELATED APPLICATIONS

This application claims the benefit of U.S. Provisional Application No. 62/849,782, filed May 17, 2019.

### BACKGROUND OF THE INVENTION

### 1. Field of the Invention

The invention relates generally to self-care boxes and more particularly to methods of using the contents of a therapeutic self-care box for providing the user with a therapeutic experience such as self-expression, relaxation, and/or self-discovery.

### 2. Description of the Related Art

Today, many people experience an unhealthy amount of stress due to health conditions, natural disasters, financial instability, and so on. Additionally, many people who experience an unhealthy amount of stress may not have the necessary coping mechanisms to deal with stress, thus they are unable to relax and reduce stress. An excess of stress can lead to health problems, as is known of ordinary skill in the art, which can lead to more stress. Some individuals may also want to have a therapeutic experience or an experience of self-expression or relaxation regardless of stress level.

Thus, there is a need to provide stressed individuals or any other individuals with a therapeutic method, which they can use to relax and reduce stress, and promote therapeutic experiences.

The aspects or the problems and the associated solutions presented in this section could be or could have been pursued; they are not necessarily approaches that have been previously conceived or pursued. Therefore, unless otherwise indicated, it should not be assumed that any of the approaches presented in this section qualify as prior art merely by virtue of their presence in this section of the application.

### BRIEF SUMMARY OF THE INVENTION

This Summary is provided to introduce a selection of concepts in a simplified form that are further described below in the Detailed Description.

In some embodiments, the invention provides a self-care box for providing users with a self-discovery, relaxation, or self-expression experience, wherein the self-care box comprises: a container for holding components used to provide the self-discovery, relaxation or self-expression experience, a canvas, a set of paints of different colors, a palette, an easel, one or more paint brushes, a journal, one or more essential oils; and a mantra sheet. The self-care box can, in some embodiments, also include a candle. The self-care box can also include written directions for using the contents of the self-care box for self-expression, relaxation, or self-discovery. The self-care box can also contain an audio recording of soothing music, a voice-guided meditation program, or both music and a voice-guided meditation program. Alternatively, the written instructions may provide the user with a url to a web site at which the user may access a voice-guided mediation program, soothing music, or both. The self-care box can also include tea, which may be contained in one or more tea bags or boxes. In some embodiments, the contents of the self-care boxes of the invention are labeled to indicate a suggested symbolism of the component. The label may be attached directly to the component, or the component may be enclosed in a box or wrapper to which the label is affixed. The self-care box can also contain, in some embodiments, a gratitude journal.

In an aspect, a self-care box can include therapeutic contents that may provide a user with a relaxing or therapeutic experience. The therapeutic contents of the self-care box may be a journal, canvas, paints, easel, paint tray, paintbrushes, essential oil, candle, and teas, for example.

The invention also provides methods of using a self-care box for purposes such as self-expression, relaxation, or self-discovery, wherein the self-care box comprises: a container for holding components used to provide the self-discovery, relaxation or self-expression experience, a canvas, a set of paints of different colors, a palette an easel, one or more paint brushes, a journal, one or more essential oils, and a mantra sheet. The method for self-expression, relaxation, or self-discovery, includes the steps of: unpacking the contents of the self-care box, lighting a candle; placing a few drops of essential oil on the user's hands, cupping the user's hands around their mouth and nose and breathing deeply, writing the user's thoughts and feelings in the journal, setting the canvas on the easel, and the paints on the palette, and preparing to paint, reading the mantra aloud, visualizing each line; listening to soothing music, a voice-guided meditation program, or a combination of both, using the paints, paint brush, and palette to paint whatever comes into the user's mind on the canvas, writing the user's thoughts and feelings in the journal a second time. The method can also include writing things for which the user is grateful on any suitable medium, for example, in a gratitude journal.

The invention also provides, in some embodiments, a “multi-user” or “family pack” self-care box that includes materials for more than one user to experience the self-discovery, relaxation and/or self-discovery methods for which the self-care box may be used. For example, the multi-user self-care box can include a plurality of easels, canvasses, palettes, and the like.

In an aspect, a user may remove the therapeutic contents in the following order: (1) the journal, (2) the canvas, (3) the paints, (4) the easel, (5) the paint tray, (6) the paintbrushes, (7) the essential oil, (8) the candle, (9) teas.

In another aspect, a user may be provided with the following suggested order and method of use of the contents of a self-care box: journal, canvas, mantra, easel, paintbrushes, candle, teas, oils, paints, and palette.

In another aspect, the contents of the self-care box may provide users with a method for relaxation and therapeutic experience via journaling and painting, for example.

Thus, an advantage may be that the self-care box may help a user to achieve a relaxed state. Another advantage may be that the instructions for unpacking and using the self-care box may be a guide for a user to maximize the therapeutic benefits of the contents of the self-care box. Another advantage may be that the contents of the self-care box may help a user on a journey of self-discovery and self-expression.

The above aspects or examples and advantages, as well as other aspects or examples and advantages, will become apparent from the ensuing description and accompanying drawings.

### BRIEF DESCRIPTION OF THE DRAWINGS

For exemplification purposes, and not for limitation purposes, aspects, embodiments or examples of the invention are illustrated in the figures of the accompanying drawings, in which:

FIG. 1 illustrates a top view of a self-care box in a closed state, according to an aspect.

FIG. 2 illustrates a top view of a self-care box in an open state, according to an aspect.

FIG. 3 illustrates a top view of a self-care box in an open state, wherein the directions have been removed, according to an aspect.

FIG. 4 illustrates a top perspective view of a self-care box in an open state, having a journal at the top interior portion of the self-care box, according to an aspect.

FIG. 5 illustrates a top perspective view of a self-care box in an open state, having a canvas at a center interior portion of the self-care box, according to an aspect.

FIG. 6 illustrates a top view of a self-care box in an open state, having paints provided at the bottom interior portion of the self-care box, according to an aspect.

FIG. 7 illustrates a top view of a self-care box in an open state, having an easel at a center interior portion of the self-care box, according to an aspect.

FIGS. 8A-8B illustrates a top perspective view of a self-care box in an open state, having a paint tray and paintbrushes within the interior space of the self-care box, and a top view of the paint tray, respectively, according to an aspect.

FIG. 9A illustrates a partial top view of a self-care box in an open state, having small boxes, according to an aspect.

FIG. 9B illustrates a top perspective view of an essential oil blend and a candle, according to an aspect.

FIG. 10 illustrates a partial top view of a self-care box in an open state, having tea boxes within the interior space of the box, according to an aspect.

FIG. 11 illustrates a perspective view of an exemplary final setup of the contents of a self-care box when using a method of using the self-care box, according to an aspect.

FIGS. 12A-12B show an example of an instruction sheet that includes directions for how to use the self-care box for purposes of self-expression, relaxation, and/or self-discovery.

### DETAILED DESCRIPTION OF THE INVENTION

What follows is a description of various aspects, embodiments and/or examples in which the invention may be practiced. Reference will be made to the attached drawings, and the information included in the drawings is part of this detailed description. The aspects, embodiments and/or examples described herein are presented for exemplification purposes, and not for limitation purposes. It should be understood that structural and/or logical modifications could be made by someone of ordinary skills in the art without departing from the scope of the invention.

It should be understood that, for clarity of the drawings and of the specification, some or all details about some structural components or steps that are known in the art are not shown or described if they are not necessary for the invention to be understood by one of ordinary skills in the art.

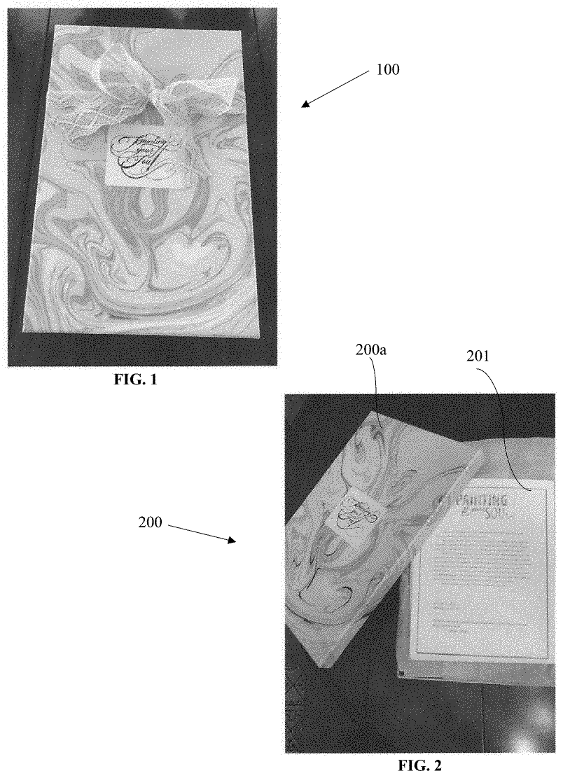

FIG. 1 illustrates a top view of a self-care box (“therapeutic box,” “self-care box,” “self-care kit,” “self-care box,” or “box”) 100 in a closed state, according to an aspect. The purpose of a self-care box may be to provide users with a therapeutic experience via relaxation and self-discovery, for example. More particularly, the self-care box may be assembled and packaged with a particular set of contents so as to facilitate a therapeutic experience when the box is unpacked and the contents of the self-care box 100 are used in a specific order by a user. A method of use of the self-care box 100 and its contents may therefore be provided to a user. As shown, a soothing pattern or other soothing packaging may be provided, such that the entire experience of having and opening a self-care box may be therapeutic.

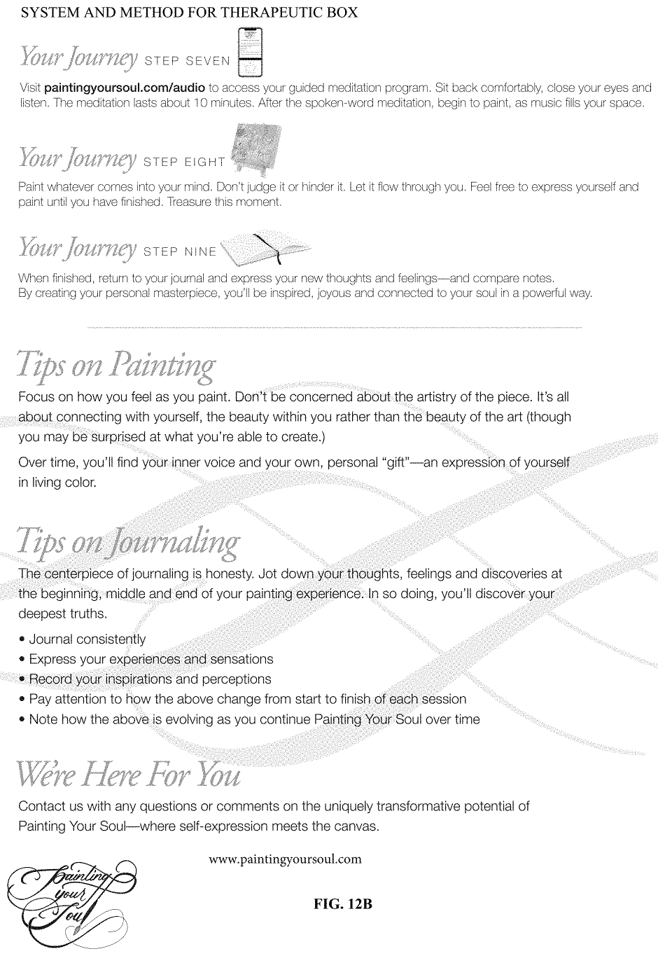

FIG. 2 illustrates a top view of a self-care box 200 in an open state, according to an aspect. As a first step in a method of using a self-care box 200, a user may remove the lid 200a and read the provided instructions 201 (see also, FIGS. 12A and 12B). Upon opening the box, the first item a user views may be the instructions 201. The instructions 201 may provide a suggested method of unpacking and use of the contents, order of use, a mindset a user can employ to achieve a maximally beneficial therapeutic experience, and symbolism of the contents of the self-care box 200, for example. It should be understood that the instructions 201 may include one or more pages. Additionally, a second document (not shown), may contain suggested mantras (e.g., affirmations) for a user. The mantras may be presented to a user at this stage in the unpacking process. As an example, suggested mantras may be: “I live fully in each moment;” “I am in touch with my inner being;” “I create abundance with ease;” and so on, or any other suitable mantra for promoting a therapeutic or relaxing experience for a user. It should be understood that the suggested mantra may be provided with the written instructions 201, or may be provided separately such as in a frame or as a magnet, for example. An advantage may be that the user may be able to more easily view the mantra while painting, or may be more easily able to separate out and notice the mantra, and more easily understand the significance of the mantra.

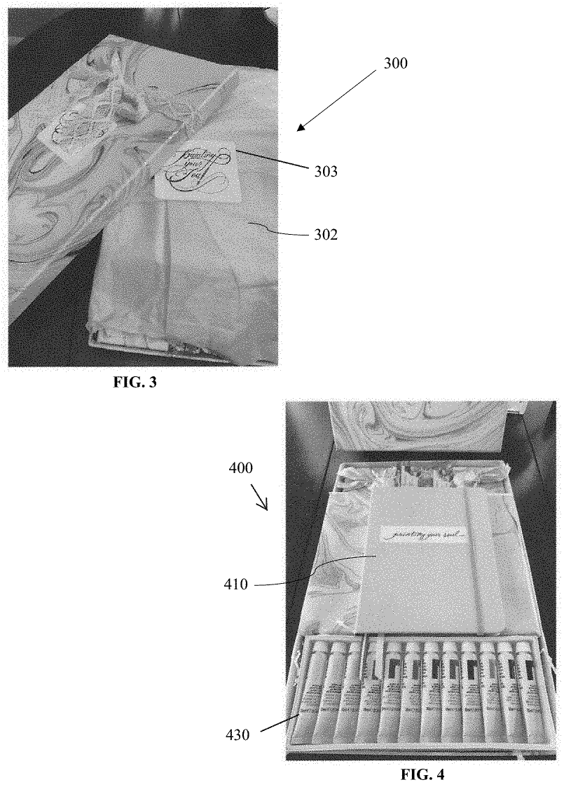

FIG. 3 illustrates a top view of a self-care box 300 in an open state, wherein the directions 201 of FIG. 2 have been removed, according to an aspect. After the instructions (shown by 201 in FIG. 2) are removed, the remaining contents of a self-care box 300 may be visible, and may be wrapped in tissue paper 302, for example. The tissue paper 302 may be selected or provided to have a texture, pattern, color, or other features to help facilitate or enhance the therapeutic experience for a user. A means for attaching or securing the tissue paper together around the contents of the box 300 may be provided, which may, for example, be a sticker 303 as shown. The sticker 303 may be provided with a logo or any other suitable decorative element or information. Other examples of a securing means may include ties or ribbons. As a second step in a method of using a self-care box 300, the user may remove the securing means 303 and the tissue paper 302.

FIG. 4 illustrates a top perspective view of a self-care box 400 in an open state, having a journal 410 at the top interior portion of the self-care box 400, according to an aspect. Next in the method of using a self-care box 400, the user may begin unpacking the contents of the self-care box 400. As a third step in a method of using a self-care box 400, the user may unpack or remove from the self-care box 400 the journal 410, for example. A paint set (“paint set,” or “paints”) 430 may also be visible in this view, at the bottom interior portion of the self-care box 400. The user may be instructed to first remove the journal 410 by the provided instructions (shown by 201 in FIG. 2). The purpose for removing the journal 410 before the other therapeutic contents is to present a user with the opportunity to discover what they are currently experiencing emotionally and physically, for example. Additionally, unpacking the journal 410 before the other therapeutic contents allows a user to utilize the journal 410 to discover personal insights during and after the unpacking process, and before, during, and after the painting process. The user may be instructed that the journal 410 could represent or symbolize “discovery,” such as discovery of personal insights before, during, and after painting.

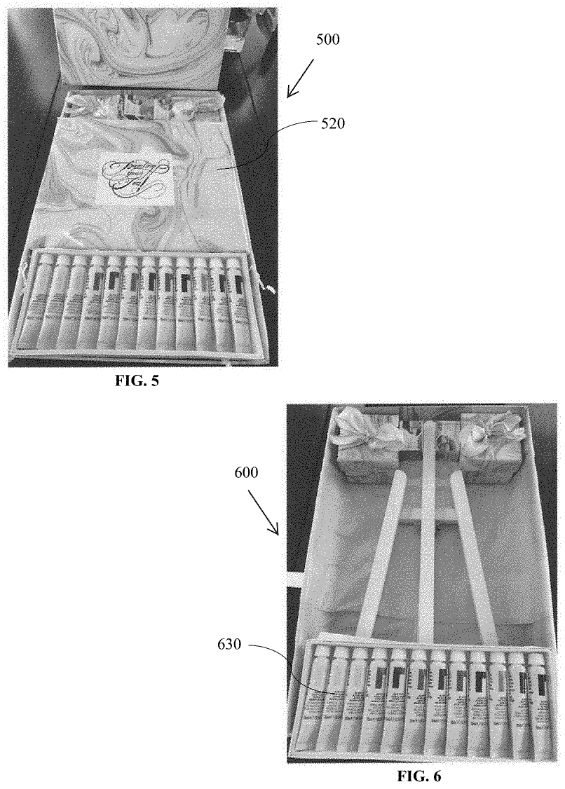

FIG. 5 illustrates a top perspective view of a self-care box 500 in an open state, having a canvas 520 at a center interior portion of the self-care box 500, according to an aspect. Next, as a fourth step in a method of using a self-care box 500, a user may remove and unwrap the canvas 520 from the box. As shown, the canvas 520 may be wrapped with a soothing wrapping paper such that the wrapping may enhance the therapeutic process for the user. The canvas 520 may serve as a type of symbolic “mirror,” for a user, which the user can use to reflect their emotional state. As another example, the user may be informed that the canvas 520 could represent their life, while the painting that the user creates on the canvas 520 could represent their journey. It should be understood that any other additional canvases may also be included within the self-care box 500.

FIG. 6 illustrates a top view of a self-care box 600 in an open state, having paints 630 provided at the bottom interior portion of the self-care box 600, according to an aspect. Next, as a fifth step in a method of using a self-care box 600, the user may remove and unpack the paints 630 from the self-care box 600. The paints 630 may serve as a way for a user to express their mindset (i.e., emotional or physical state, personal insights, etc.) via visual stimulation, and the act of painting. The user may also be instructed that the paints 630 could symbolize “soul colors,” and be representative of the brilliance within the person using the self-care box 600.

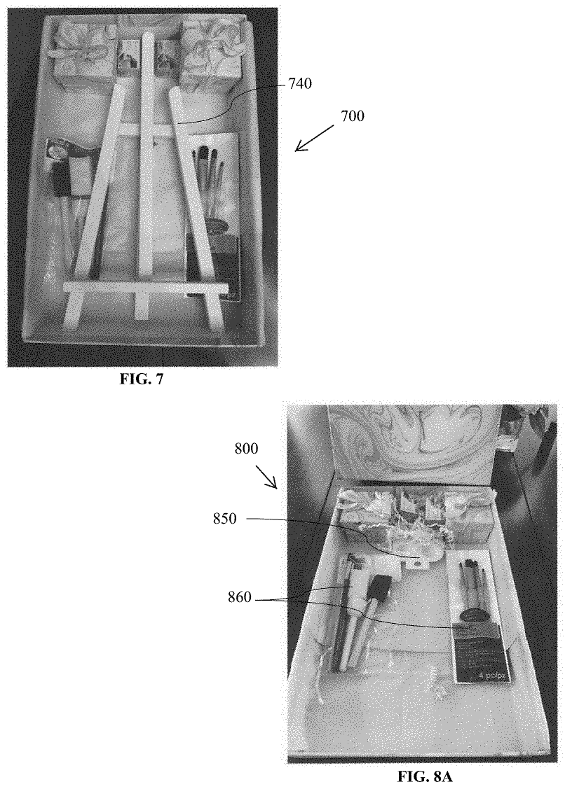

FIG. 7 illustrates a top view of a self-care box 700 in an open state, having an easel 740 at a center interior portion of the self-care box 700, according to an aspect. Next, as a sixth step in a method of using a self-care box 700, a user may remove the easel 740 from the self-care box 700. The user may also be informed that the easel could symbolize “strength.” More particularly, the easel 740 may hold the canvas (shown by 520 in FIG. 5) securely so as to help a user remember that they are not alone, and the user can gain strength and support from others.

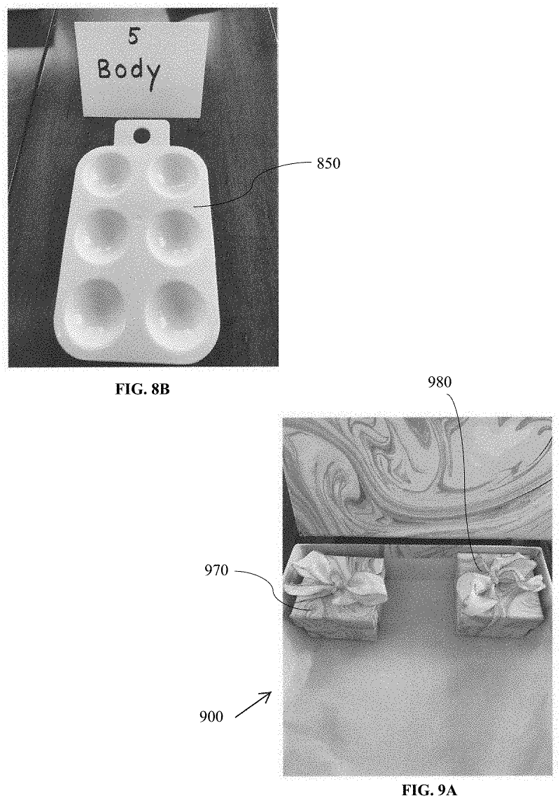

It should be noted that, while the paint tray (shown by 850 in FIG. 8a) is not shown in FIG. 7 for visual clarity, the paint tray may not yet be removed at this step in the unpacking process.

FIGS. 8A-8B illustrates a top perspective view of a self-care box 800 in an open state, having a paint tray 850 and paintbrushes 860 within the interior space of the self-care box 800, and a top view of the paint tray 850, respectively, according to an aspect. Next, as a seventh step in a method of using a self-care box 800, a user may remove the paint tray 850 from the box 800. The user may also be instructed that the paint tray 850 could represent “body,” by holding the beautiful colors of the soul of the user, and the paint tray 850 may also hold the paints 630 as a “body” holds a user's emotions, which are expressed via the act of painting and the resulting painting.

Next, as an eighth step in a method of using a self-care box 800, a user may remove the paint brushes 860 from the box 800. The paintbrushes 860 may serve as a way for a user to connect and channel their emotions to the canvas (shown by 520 in FIG. 5), such that the user may more easily express their emotions via painting on a canvas and achieve a therapeutic experience. The user may be instructed that the paintbrushes 860 could symbolize or represent a “heart link,” linking the user's heart to the canvas.

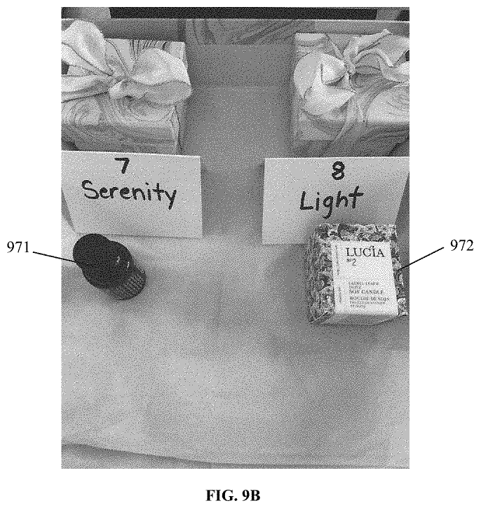

FIG. 9A illustrates a partial top view of a self-care box 900 in an open state, having small boxes 970 and 980, according to an aspect. The small boxes may be a first small box 970 and a second small box 980. The first small box 970 may be packed or provided in an upper left corner of the interior space of the self-care box 900, and the second small box 980 may be packed or provided in an upper right corner of the interior space of the self-care box 900. As a ninth step in a method of using a self-care box 900, the user may remove both the first small box 970 and the second small box 980. As shown as an example, the small boxes 970 and 980 may be provided with the same or similar soothing pattern or color as the self-care box (as shown by 100 in FIG. 1), for example.

FIG. 9B illustrates a top perspective view of an essential oil blend 971 and a candle 972, according to an aspect. As an example, the first small box 970 may contain the essential oil blend 971, which may be packaged in a bottle, and which may serve the purpose of helping a user to fully relax and become immersed in a therapeutic experience via breathing an aromatherapy scent. The user may be instructed that the essential oil blend could represent “serenity,” and represent breathing in peace and love. As another example, the second small box 980 may contain a candle 972, which may be made of soy wax, and may further be provided in its own packaging. The purpose of the candle 972 within the second small box 980 may be to further immerse a user into a therapeutic experience, by providing a soothing light source. The user may be instructed that the candle could represent “light,” such as the user's “inner light.” It should be understood that the essential oil blend 971 and the candle 972 may be provided without individual packaging, and may be placed directly in the self-care box, or may be placed in a pouch within the self-care box.

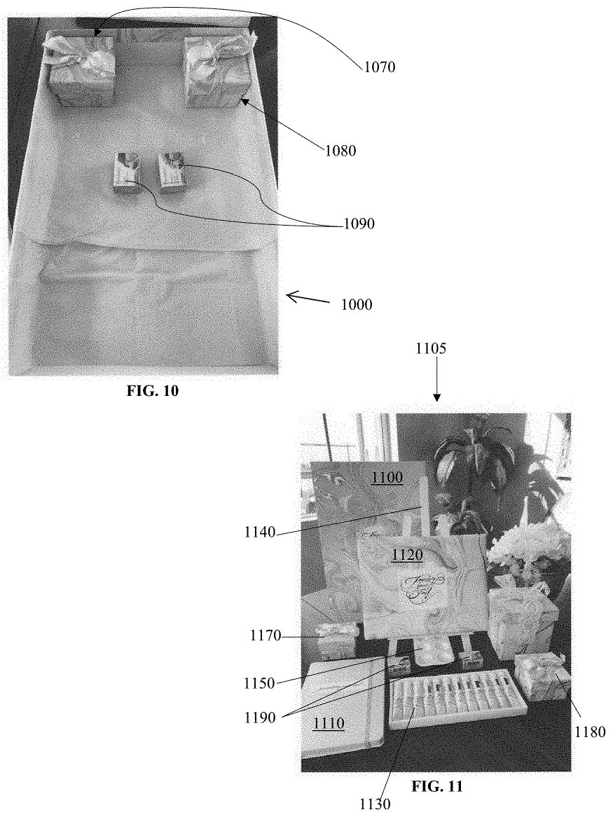

FIG. 10 illustrates a partial top view of a self-care box 1000 in an open state, having tea boxes 1090 within the interior space of the box 1000, according to an aspect. Next, as an eleventh step in a method of using a self-care box 1000, the user may remove the tea boxes 1090. Two tea boxes 1090 may be provided. It should be understood that any other suitable number of tea boxes may be used or provided. As an example, both of the teas contained in the two tea boxes 1090 may be herbal teas, which may soothe a user during their therapeutic experience, and help the user to become relaxed. The user may also be instructed that the tea may be represent or symbolize “taste,” for soothing the user along the way or process of using the self-care box 1000. The first small box 1070 and the second small box 1080 may also be visible in this view.

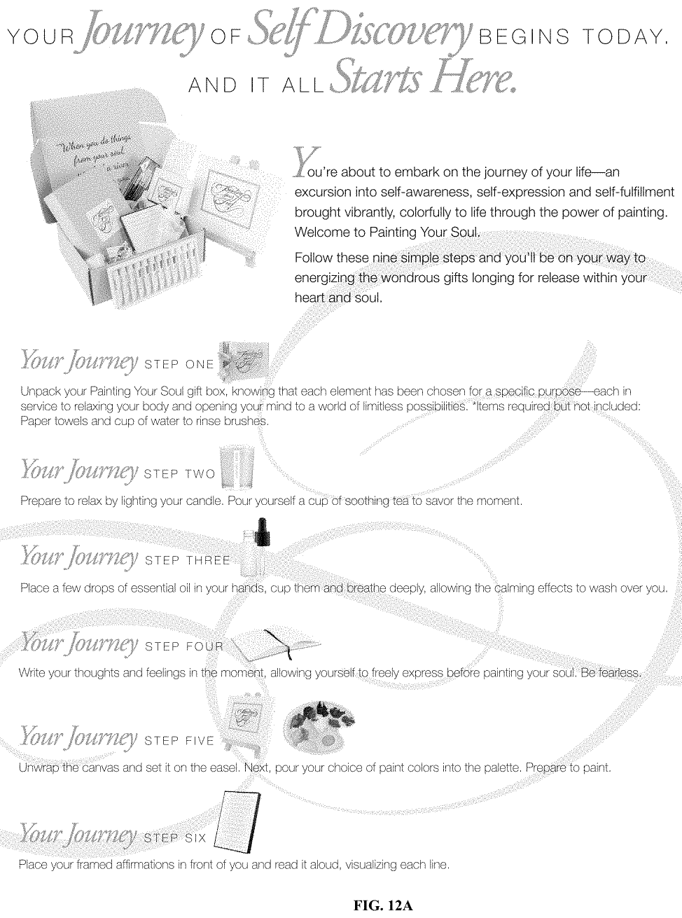

FIG. 11 illustrates a perspective view of an exemplary final setup 1105 of the contents of a self-care box when using a method of using the self-care box shown and described when referring to FIGS. 3-10, according to an aspect. The exemplary final setup 1105 shown in FIG. 11 may be an optimal arrangement to facilitate a therapeutic experience, for example. Again, the therapeutic contents of the self-care box 1100 may be removed then setup as shown in FIG. 11 in the following order: (1) the journal 1110, (2) the canvas 1120, (3) the paints 1130, (4) the easel 1140, (5) the paint tray 1150, (6) the paint brushes (not shown in FIG. 11), (7) the first small box 1170 containing essential oil, (8) the second small box 1180 containing a candle, (9) teas 1190. As an example, the paintbrushes (shown by 860 in FIG. 8A) may be placed in front of the paints 1130. As another example, an additional, optional box 1180 is also shown in this view, which may be provided with the self-care box for housing additional contents or any other suitable gifts or objects. As another example, a pen may be included with the self-care box, which may be advantageous for the user such that they can more easily and quickly use the provided journal 1110.

FIGS. 12A and 12B show an example of written instructions for using the contents of a self-care box of the invention for self-expression, relaxation, and/or self-discovery.

The following is an exemplary process disclosing how a user may utilize the contents of the self-care box.

First, a user may open the journal 1110 and prepare to write down whatever comes to their mind. Next, a user may unwrap their canvas 1120 and place the canvas 1120 on the easel 1140. Now, a user can light the provided candle. Then, a user may place a mantra sheet in front of the canvas 1120 on the easel 1140. A user may then read the mantra sheet quietly, such that they can visualize and internalize each line of the mantra sheet. Next, a user can take the essential oil blend and place a few drops on their hands, such that the user can cup their hands around their mouth and nose and breathe deeply, so as to fully experience the aromatherapy benefits provided by the essential oil. Next, a user can listen to soothing audio, which may be provided in addition to the contents of the self-care box, for example, or provided by a web site that the user may access through use of a provided url. As an example, the provided audio may be soothing music or a guided meditation, which may last for a duration of seven minutes, for example. As the provided audio plays and further assists a user to achieve a relaxing mindset, a user can begin journaling and writing down whatever comes to mind.

After a user feels they have completed journaling, the user can then use the provided canvas, paint, paintbrushes, and paint tray to begin painting and engage in self-expression. Audio may be provided to enhance the user's therapeutic experience, which may last for a duration of sixty minutes, for example. Again, the audio, which may include music, guided meditation, or both, may be provided through access to a web site using a url that is provided in the therapeutic box to the user.

Once a user is done painting, the use can once again begin journaling. Journaling before and after painting allows a user to compare their emotional and physical state as well as their personal insights before and after the therapeutic painting process. Journaling may continue after the initial painting session, and the user may continue journaling as desired or suggested by a provider of the self-care box.

It should be understood that the contents of the self-care box may be arranged in a more or less compact manner according to the size and shape of the box provided. It should also be understood that any suitable labels may be used for labeling the various components of the self-care box such that a user may more easily visualize and understood the suggested order of unpacking and use of the contents. The labels may also indicate the suggested symbolism of each component of the self-care box. It should also be understood that other suggested methods of use, suggested orders of use and unpacking, or any other suitable directions or instructions may also be provided with the contents of the self-care box, such that a relaxing or therapeutic experience for the user may be promoted or encouraged.

The invention also provides methods for achieving one or more of enhanced self-expression, self-awareness, self-fulfillment and relaxation. These methods can involve, for example, (a) writing one's thoughts and feelings in a journal, (b) reading one or more affirmations, (c) painting, and (d), after painting, writing one's thoughts and feelings in the journal. The methods can also include lighting a candle prior to step (a). In some embodiments, the methods can also involve, before step (a), placing essential oil on the user's hands, cupping the user's hands around their nose and mouth, and breathing in the scents of the essential oil(s). Both lighting a candle and breathing in the essential oil scent are performed before step (a) in some embodiments.

The methods can also involve listening to a guided meditation program. The guided meditation program can include spoken instructions for performing the methods of the invention. The program can also include phrases that assist the user in embarking on a visual journey, in the mind's eye, using colors to express the deepest part of one's being. The program may also assist the user in developing their own personal rhythm, placing the user in the present moment but in a state of flow, developing a deep connection to the user's self, discovering how magnificent and beautiful the user is. Listening to this portion of the guided program is intended to prepare the user to paint, without any fear, judgement, anxiety, or inhibitions.

In some embodiments, the methods can also include writing down things for which the user is grateful. The user may write the items on any suitable writing medium. One example of a suitable medium is a gratitude journal.

It may be advantageous to set forth definitions of certain words and phrases used in this patent document. The term “couple” and its derivatives refer to any direct or indirect communication between two or more elements, whether or not those elements are in physical contact with one another. The term “or” is inclusive, meaning and/or. As used in this application, “and/or” means that the listed items are alternatives, but the alternatives also include any combination of the listed items.

The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

Further, as used in this application, “plurality” means two or more. A “set” of items may include one or more of such items. The terms “comprising,” “including,” “carrying,” “having,” “containing,” “involving,” and the like are to be understood to be open-ended, i.e., to mean including but not limited to. Only the transitional phrases “consisting of” and “consisting essentially of,” respectively, are closed or semi-closed transitional phrases.

Throughout this description, the aspects, embodiments or examples shown should be considered as exemplars, rather than limitations on the apparatus or procedures disclosed. Although some of the examples may involve specific combinations of method acts or system elements, it should be understood that those acts and those elements may be combined in other ways to accomplish the same objectives.

Acts, elements and features discussed only in connection with one aspect, embodiment or example are not intended to be excluded from a similar role(s) in other aspects, embodiments or examples.

Aspects, embodiments or examples of the invention may be described as processes, which are usually depicted using a flowchart, a flow diagram, a structure diagram, or a block diagram. Although a flowchart may depict the operations as a sequential process, many of the operations can be performed in parallel or concurrently. In addition, the order of the operations may be re-arranged. With regard to flowcharts, it should be understood that additional and fewer steps may be taken, and the steps as shown may be combined or further refined to achieve the described methods.

Although aspects, embodiments and/or examples have been illustrated and described herein, someone of ordinary skills in the art will easily detect alternate of the same and/or equivalent variations, which may be capable of achieving the same results, and which may be substituted for the aspects, embodiments and/or examples illustrated and described herein, without departing from the scope of the invention. Therefore, the scope of this application is intended to cover such alternate aspects, embodiments and/or examples.

1. A self-care box for providing users with a self-discovery, relaxation, or self-expression experience, wherein the self-care box comprises:
a) a container for holding components used to provide the self-discovery, relaxation or self-expression experience;
b) a canvas;
c) a set of paints of different colors;
d) a palette;
e) an easel;
f) one or more paint brushes;
g) a journal;
h) one or more essential oils; and
i) a mantra sheet.

2. The self-care box of claim 1, wherein the self-care box further comprises a candle.

3. The self-care box of claim 1, wherein the self-care box further comprises an audio recording of soothing music, a voice-guided meditation program, or both music and a voice-guided meditation program.

4. The self-care box of claim 1, wherein the self-care box further comprises written directions for using the contents of the self-care box for self-expression, relaxation, or self-discovery.

5. The self-care box of claim 4, wherein the written instructions provide a url for accessing a voice-guided meditation program.

6. The self-care box of claim 1, wherein the self-care box further comprises one or more tea bags or boxes.

7. The self-care box of claim 1, wherein the essential oils are a blend of essential oils.

8. The self-care box of claim 1, wherein one or more of the contents of the self-care box are labeled to indicate a suggested symbolism of the component.

9. The self-care box of claim 8, wherein the label is affixed to a box or wrapper that contains the component.

10. The self-care box of claim 1, wherein the self-care box further comprises a gratitude journal.

11. A method of using a self-care box for self-expression, relaxation, or self-discovery, wherein:
a) the self-care box comprises:
i) a container for holding components used to provide the self-discovery, relaxation or self-expression experience;
ii) a canvas;
iii) a set of paints of different colors;
iv) a palette;
v) an easel;
vi) one or more paint brushes;
vii) a journal;
viii) one or more essential oils; and
ix) a mantra sheet.
b) wherein a user performs the method for self-expression, relaxation, or self-discovery, which method comprises the steps of:
i) unpacking the contents of the self-care box;
ii) lighting a candle;
iii) placing a few drops of essential oil on the user's hands, cupping the user's hands around their mouth and nose and breathing deeply;
iv) writing the user's thoughts and feelings in the journal;
v) setting the canvas on the easel, and one or more of the paints on the palette, and preparing to paint;
vi) reading the mantra(s) aloud, visualizing each line;
vii) listening to soothing music, a voice-guided meditation program, or a combination of both;
viii) using the paints, paint brush, and palette to paint whatever comes into the user's mind on the canvas;
ix) writing the user's thoughts and feelings in the journal a second time.

12. The method of claim 11, wherein the self-care box further comprises a candle.

13. The method of claim 11, wherein the self-care box further comprises written instructions for using the contents of the self-care box for self-expression, relaxation, or self-discovery.

14. The method of claim 11, wherein the self-care box further comprises a gratitude journal and the method further comprises writing things for which the user is grateful in the gratitude journal.

15. The method of claim 11, wherein the soothing music, voice-guided meditation program, or both, are provided by accessing an external web site using a url provided with the self-care box.

16. A method for achieving one or more of enhanced self-awareness, self-expression, self-discovery, and relaxation, the method comprising:
a) writing one's thoughts and feelings in a journal;
b) reading one or more affirmations;
c) painting; and
d) after painting, writing one's thoughts and feelings in the journal.

17. The method of claim 16, wherein the method further comprises lighting a candle before step (a).

18. The method of claim 16, wherein the method further comprises, before step (a), placing essential oil on the user's hands, cupping the user's hands around their nose and mouth, and breathing deeply.

19. The method of claim 16, wherein the method further comprises listening to a guided meditation program.
